(19) United States
(12) Patent Application Publication
(10) Pub. No.: US 2016/0058621 A1
(43) Pub. Date: Mar. 3, 2016

(54) WOUND DRESSING

(71) Applicant: Guangdong Meiji Biotechnology Co., Ltd., Guangzhou (CN)

(72) Inventor: Xinsheng ZHU, Guangzhou (CN)

(21) Appl. No.: 14/622,802

(22) Filed: Feb. 13, 2015

Related U.S. Application Data
(63) Continuation-in-part of application No. PCT/CN2014/094397, filed on Dec. 19, 2014.

(30) Foreign Application Priority Data
Aug. 29, 2014 (CN) 201410438782.7

(51) Int. Cl. A61F 13/00; A61L 26/00

(52) U.S. Cl. CPC A61F 13/00042; A61F 13/00017; A61L 26/00

(57) ABSTRACT

A wound dressing including a sheet body made of a compressed polyvinyl alcohol foam material. The sheet body includes a liquid absorption and locking layer in the form of a three-dimensional intercommunicating network, two liquid-permeable layers disposed at two sides of the liquid absorption and locking layer, respectively, and a plurality of liquid absorption holes running through the sheet body. The liquid-permeable layers are a one-way permeable membrane for preventing fluid loss.

### CROSS-REFERENCE TO RELATED APPLICATIONS

This application is a continuation-in-part of International Patent Application No. PCT/CN2014/094397 with an international filing date of Dec. 19, 2014, designating the United States, now pending, and further claims priority benefits to Chinese Patent Application No. 201410438782.7 filed Aug. 29, 2014. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND OF THE INVENTION

1. Field of the Invention

The invention relates to a wound dressing.

2. Description of the Related Art

Exudate or blood from wounds contains proteins and other suspended substances. Existing medical gauze can preferentially absorb water and the proteins are caught between the wound and the dressing. There, they form good culture medium for bacteria, which poses a serious risk of infection.

### SUMMARY OF THE INVENTION

In view of the above-described problems, it is one objective of the invention to provide a wound dressing that can absorb viscous exudates and blood completely thereby reducing the risk of infection, and shortening the healing time of the wounds.

To achieve the above objective, in accordance with one embodiment of the invention, there is provided a wound dressing comprising a sheet body being a compressed polyvinyl alcohol foam material, the sheet body comprising a liquid absorption and locking layer in the form of a three-dimensional intercommunicating network, two liquid-permeable layers disposed at two sides of the liquid absorption and locking layer, respectively, and a plurality of liquid absorption holes running through the sheet body, wherein the liquid-permeable layers are a one-way permeable membrane for preventing fluid loss, and the three-dimensional intercommunicating network is filled with epoxy resin powder for locking water.

In a class of this embodiment, the sheet body comprises a plurality of inspection windows.

In a class of this embodiment, the liquid absorption and locking layer is a porous sponge layer.

In a class of this embodiment, the liquid absorption holes have a pore size of between 0.5 and 1.5 mm, which is less than that of the inspection window; the liquid absorption holes are circular, elliptical, triangular, trapezoidal, semicircular or semi-elliptical.

In a class of this embodiment, a cross section of the inspection window is round, rectangular, oval, circular, trapezoidal, semicircular, semi-oval, or triangular.

Advantages according to embodiments of the invention are summarized as follows. In use, the wound dressing is attached to the wound by a transparent medical viscose. When the wound dressing contacts with the surface of wound, the compressed polyvinyl alcohol restores from the compressed status to the expansion status, the absorption capacity is produced, a pressure difference is generated between the liquid absorption and locking layer and the surface of wound, so that the exudate and blood drains from the surface of wound to the liquid absorption and locking layer. The liquid absorption and locking layer has strong hydrophilicity and liquid absorption and locking ability, and can absorb and lock the exudate and blood. In addition, the drainage of the exudate and blood promotes the formation of local micro negative pressure, which alleviates the tissue swelling, promotes the blood circulation, induces the granulation or epithelial cell growth, and accelerates the healing of the wounds. The liquid-permeable layers are a semipermeable membrane, thus, on one hand, the moist microenvironment of the wound is maintained; on the other hand, the exudate accumulation of the wound is avoided. No residues of solid exudate such as proteins accumulate on the surface of the wound, so the bacteria cannot propagate on the wound, thereby greatly reducing the risk of infection of the wound and shortening the healing time.

The sheet body comprises a plurality of inspection windows which are unobstructed all the time, through which, the healing status of the wounds are observed at any time around the clock. Based on the observation results, it can be determined whether the wound dressing need replacing, thereby avoiding unnecessary replacing, saving the use costs, and alleviating the pain of patients. The inspection windows are formed on the sheet body by laser drilling or punching.

### BRIEF DESCRIPTION OF THE DRAWINGS

The invention is described hereinbelow with reference to the accompanying drawings, in which:

FIG. 1 is an axonometric drawing of a wound dressing according to one embodiment of the invention;

FIG. 2 is a top view of a wound dressing according to one embodiment of the invention; and

FIG. 3 is a sectional view of a wound dressing according to one embodiment of the invention.

### DETAILED DESCRIPTION OF THE EMBODIMENTS

For further illustrating the invention, experiments detailing a wound dressing are described below. It should be noted that the following examples are intended to describe and not to limit the invention.

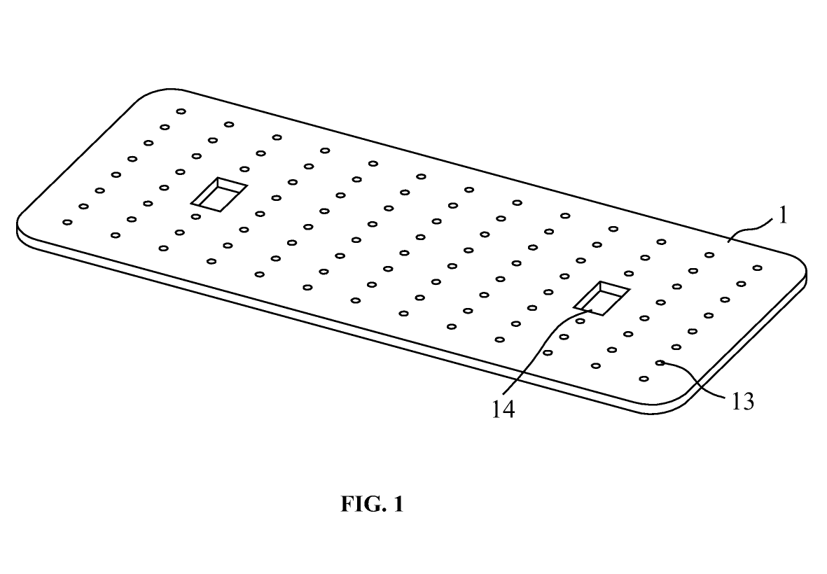

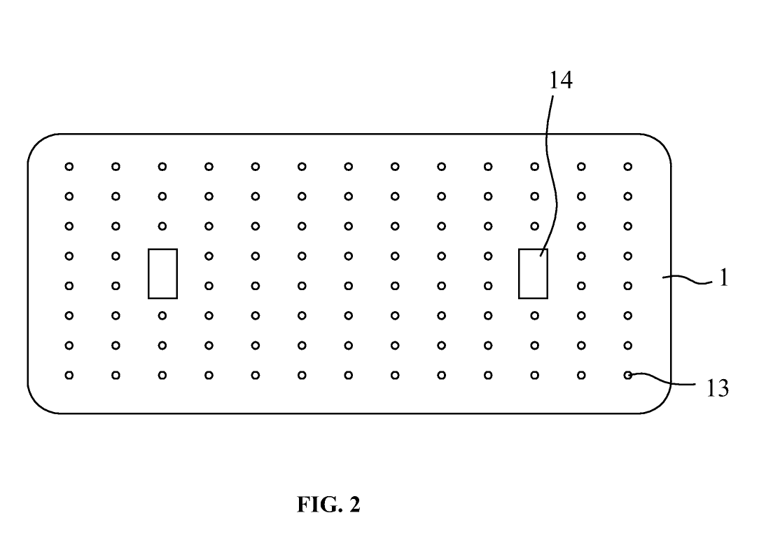

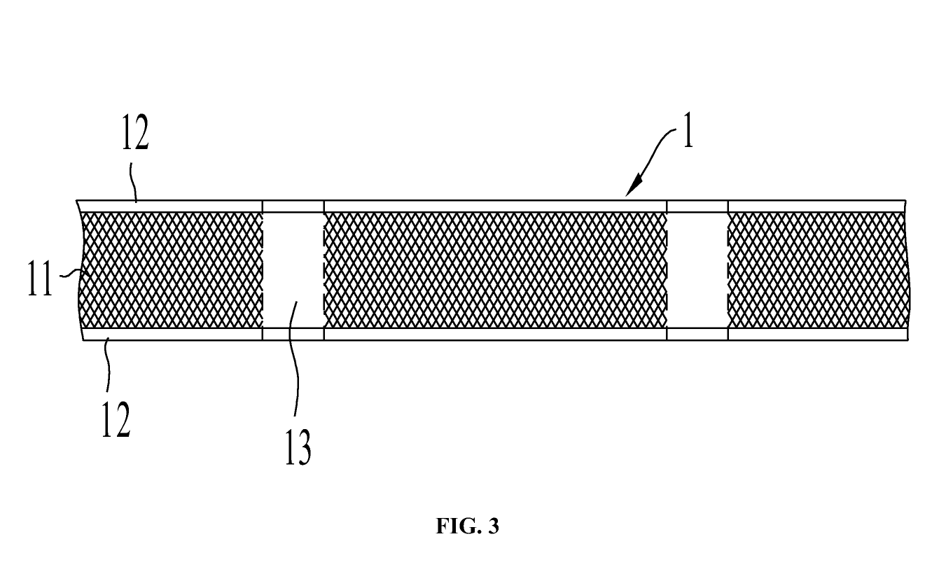

As shown in FIGS. 1-3, the invention provides a wound dressing comprising a sheet body 1 which is made of compressed polyvinyl alcohol foam material. The sheet body comprises a liquid absorption and locking layer 11 in the form of a three-dimensional intercommunicating network, two liquid-permeable layers 12 disposed at two sides of the liquid absorption and locking layer, respectively, and a plurality of liquid absorption holes 13 running through the sheet body. The liquid absorption and locking layer 11 is a porous sponge layer. The liquid-permeable layers 12 are a one-way permeable membrane for preventing fluid loss. To improve the water locking capacity, the three-dimensional intercommunicating network is filled with epoxy resin powders for locking water.

In use, the wound dressing is attached to the wound, so that the liquid absorption and locking layer 11 and the liquid absorption holes 13 absorb the exudate or blood from wounds quickly, which is beneficial to drainage of the wound, thereby promoting the local blood circulation and the concrescence of the wound. The wound dressing comprises three layered structures and absorption holes, so that the exudate and blood of the wound can be absorbed instantly by the liquid absorption and locking layer 11 and are locked in the liquid absorption and locking layer 11 by the help of the liquid-permeable layers 12. Thus, on one hand, the moist microenvironment of the wound is maintained; on the other hand, the exudate accumulation of the wound is avoided. No residues of solid exudate such as cellulose and proteins accumulate on the surface of the wound, so the bacteria cannot propagate on the wound, thereby greatly reducing the risk of infection of the wound. The application of the wound dressing enables the wound to heal without incrustation, thereby greatly shortening the healing time of the wound.

The sheet body comprises a plurality of inspection windows 14, through which, the healing status of the wounds are observed at any time around the clock. Based on the observation results, it can be determined whether the wound dressing need replacing, thereby avoiding unnecessary replacing, saving the use costs, and alleviating the pain of patients. The inspection windows 14 are formed on the sheet body by laser drilling or punching.

The pore size of the liquid absorption holes is between 0.5 and 1.5 mm, which is less than that of the inspection window. The liquid absorption holes are circular holes, elliptical holes, triangular holes, trapezoidal holes, semicircular or semi-elliptical holes. The cross section of the inspection window is round, rectangle, oval, trapezoid, semicircle, semioval, or triangle in shape, which is determined according to the shape of the surface of wounds.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A wound dressing, comprising a sheet body being a compressed polyvinyl alcohol foam material, the sheet body comprising: (a) a liquid absorption and locking layer in the form of a three-dimensional intercommunicating network, (b) two liquid-permeable layers disposed at two sides of the liquid absorption and locking layer, respectively, and (c) a plurality of liquid absorption holes running through the sheet body, wherein the liquid-permeable layers are a one-way permeable membrane for preventing fluid loss, and the three-dimensional intercommunicating network is filled with epoxy resin powder.

2. The wound dressing of claim 1, wherein the sheet body comprises a plurality of inspection windows.

3. The wound dressing of claim 1, wherein the liquid absorption and locking layer is a porous sponge layer.

4. The wound dressing of claim 2, wherein the liquid absorption holes have a pore size of between 0.5 and 1.5 mm, which is less than that of the inspection window;
the liquid absorption holes are circular, elliptical, triangular, trapezoidal, semicircular or semi-elliptical.

5. The wound dressing of claim 2, wherein a cross section of the inspection window is round, rectangular, oval, circular, trapezoidal, semicircular, semi-oval, or triangular.
